(19) United States
(12) Patent Application Publication
(10) Pub. No.: US 2019/0023683 A1
(43) Pub. Date: Jan. 24, 2019

(54) PROCESS FOR THE PREPARATION OF ALOGLIPTIN

(71) Applicant: INDOCO REMEDIES LIMITED, Mumbai, MH (IN)

(72) Inventors: Ranjeet NAIR, Navi Mumbai (IN); Palangat Vayalileveetil RAMESAN, Navi Mumbai (IN); Aditi Milind PANANDIKAR, Mumbai (IN)

(21) Appl. No.: 15/571,747

(22) PCT Filed: May 3, 2016
(86) PCT No.: PCT/IN2016/050122

§ 371 Date: Nov. 3, 2017

(30) Foreign Application Priority Data
May 4, 2015 (IN) 1772/MUM/2015

(51) Int. Cl. C07D 401/04

(52) U.S. Cl. CPC C07D 401/04; A61P 3/10

(57) ABSTRACT

The present invention discloses a novel process for the preparation of alogliptin and its pharmaceutically acceptable salt.

### FIELD OF INVENTION

The present invention relates to a novel process for the preparation of alogliptin and its pharmaceutically acceptable salt.

### BACKGROUND OF THE INVENTION

The compound, 2-[[6-[(3R)-3-amino-1-piperidinyl]-3,4-dihydro-3-methyl-2,4-dioxo-1(2H)-pyrimidinyl]methyl]benzonitrile commonly known as alogliptin represented below as the compound of Formula I, is a dipeptidyl peptidase-4 inhibitor (DPP-4) that is designed to slow the inactivation of incretin hormones glucagon-like peptide-1 (GLP-1) and glucose-dependent insulinotropic peptide (GIP).

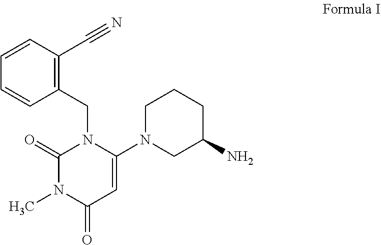 [SMILES: Cn1c(=O)cc(N2CCC[C@@H](N)C2)n(Cc2ccccc2C#N)c1=O]

DPP-4 inhibitors are a class of oral hypoglycemic that block DPP-4 and are very effective and useful for treating Type 2 diabetes mellitus. This class of anti-diabetic drugs offers some advantages over existing oral options for the management of type 2 diabetes such as a negligible risk of hypoglycemia compared with sulfonylureas and, in general, a weight-neutral profile. DPP-4 inhibitors such as sitagliptin, vildagliptin, saxagliptin, linagliptin, alogliptin and teneligliptin are currently in clinical use. These DPP-4 inhibitors are commonly known as gliptins. Like other gliptins, alogliptin causes little or no weight gain and exhibits relatively modest glucose-lowering activity. Alogliptin benzoate is sold by Takeda Pharma under the trade name Nesina® in the US, and Vipidia® in Europe.

Alogliptin and its pharmaceutical acceptable salts and process for their preparation are described in the patents, U.S. Pat. No. 7,807,689 (“US'689 patent) and its corresponding European patent, EP 1586571. The process described in US'689 patent involves reacting 6-chlorouracil with α-bromo-o-tolunitrile in the presence of strong alkali such as sodium hydride (NaH) and lithium bromide (LiBr) in a mixture of solvents such as dimethylformamide (DMF) and dimethylsulfoxide (DMSO) to yield the intermediate compound, 2-(6-chloro-2,4-dioxo-3,4-dihydro-2H-pyrimidin-1-ylmethyl)benzonitrile. The intermediate compound on methylation using methyl iodide (MeI) in the presence of sodium hydride and a mixture of solvents, dimethylformamide (DMF) and tetrahydrofuran (THF) yields N-methylated intermediate compound, 2-[(6-chloro-3-methyl-2,4-dioxo-3,4-dihydro-2H-pyrimidin-1-yl)methyl]benzonitrile. The N-methylated intermediate compound on reaction with (R)-3-amino-piperidine dihydrochloride in the presence of sodium bicarbonate and a solvent such as methanol yields alogliptin free base, the compound of Formula I. The above process for the preparation of alogliptin is schematically represented in the following Scheme I.

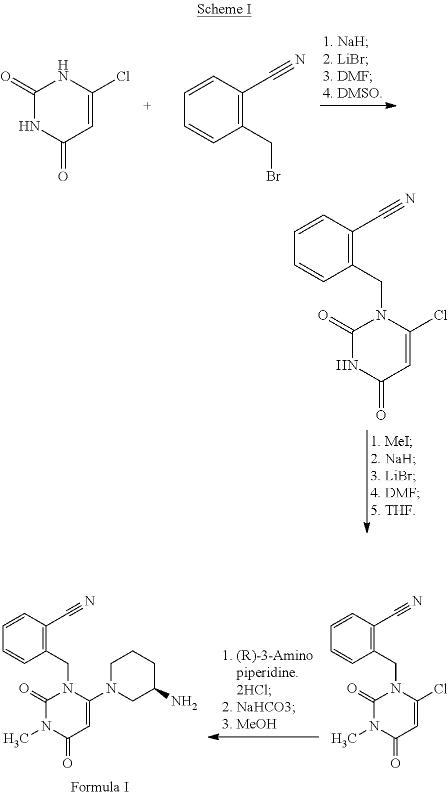 [SMILES: O=c1cc(Cl)[nH]c(=O)[nH]1 ; N#Cc1ccccc1CBr ; N#Cc1ccccc1Cn1c(Cl)cc(=O)[nH]c1=O ; Cn1c(=O)cc(N2CCC[C@@H](N)C2)n(Cc2ccccc2C#N)c1=O ; Cn1c(=O)cc(Cl)n(Cc2ccccc2C#N)c1=O]

Another patent, U.S. Pat. No. 8,222,411 describes a process for the preparation of alogliptin and its pharmaceutically acceptable salts wherein the compound 6-chloro-3-methyluracil is reacted with α-bromo-o-tolunitrile to give the intermediate compound, 2-[(6-chloro-3-methyl-2,4-dioxo-3,4-dihydro-2H-pyrimidin-1-yl)methyl]benzonitrile. The intermediate compound is then reacted with (R)-3-aminopiperidine dihydrochloride in isopropanol and water in the presence of potassium carbonate to get alogliptin hydrochloride, which on reaction with potassium carbonate followed by reaction with benzoic acid yields the benzoate salt of alogliptin, the compound of Formula I.

PCT Patent Application No. WO2010109468 describes preparation of alogliptin and its pharmaceutically acceptable salt wherein the urea derivative of compound A is reacted with malonic acid derivative or cyanoacetic acid derivative to give compound B or B1. The compound B or B1 on reaction with halogenating agent yields the compound C, 6-halopyrimidinedione, which on reaction with Boc protected (R)-3-aminopiperidine, yields protected alogliptin free base of compound D. The compound D on reaction with an acid results in corresponding acid addition salt of alogliptin. The reaction sequence is as shown in Scheme II below.

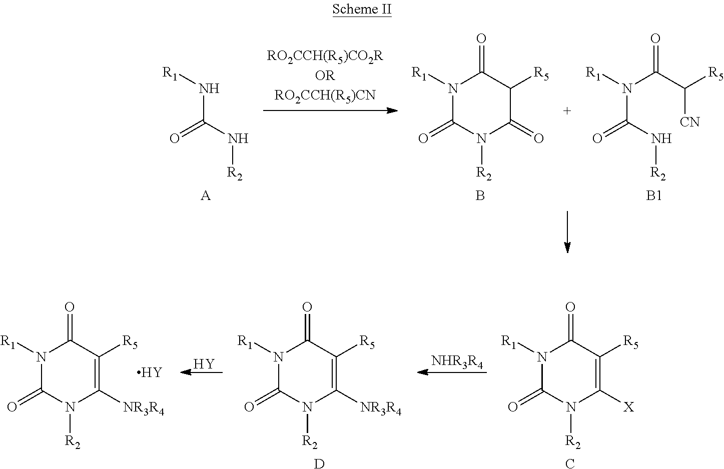

wherein: R₁ and R₅ are each independently H or (C₁-C₁₀) alkyl;
R₂ is CH₂Ar; and R₃ and R₄ together with the nitrogen to which they are attached form a 4, 5, 6 or 7 membered ring, which may be unsubstituted or substituted. Another published patent application WO2013046229 describes the preparation of amorphous alogliptin benzoate and novel acid addition salts of alogliptin i.e., fumarate, maleate, sulfate, tosylate, oxalate, nitrate, crystalline hydrochloride & crystalline tartrate.

The major drawback of the prior artprocess as described in US'689 patent is that it involve use of hazardous reagents such as sodium hydride and lithium bromide. Also, the other prior art method requires the protection of the amino compound before reacting, which results in increase in the number of steps thereby making the process cumbersome and uneconomical.

Therefore, there remains a need in the art to develop a safe and cost effective process for the preparation of alogliptin by using economical reagents which makes the process industrially viable and hence, advantageous. The present invention therefore seeks to address these issues.

Thus, it is an objective of the present invention to develop a simple and cost effective process employing industrially safe and readily available starting materials.

### SUMMARY OF THE INVENTION

Accordingly, the present invention provides a process for preparing alogliptin the compound of Formula I and its pharmaceutically acceptable salts using readily available, cost effective, and industrially safe starting materials.

According to primary object of the present invention, there is provided a simple, cost effective and industrially safe process for the preparation of alogliptin free base the compound of Formula I and its pharmaceutically acceptable salt, which comprises the steps of:
i. reacting N-methylbarbituric acid, the compound of Formula III;

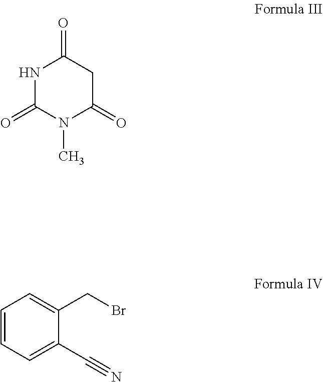

with a halogenating reagent in the presence of a base and a solvent to get an intermediate compound which is reacted in situ with 2-(bromomethyl)benzonitrile, the compound of Formula IV in the presence of a base and a second solvent to obtain 2-[(6-chloro-3-methyl-2,4-dioxo-3,4-dihydropyrimidin-1-(2H)-yl)methyl]benzonitrile, the compound of Formula V;

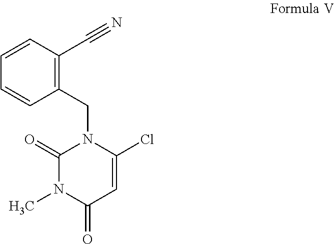 [SMILES: Cn1c(=O)cc(Cl)n(Cc2ccccc2C#N)c1=O]

ii. reacting the compound of Formula V obtained in step (a) with (R)-piperidin-3-amine free base or its salt, the compound of Formula VI

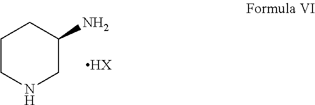

Wherein X is Cl or Br;
in the presence of an organic solvent to obtain alogliptin free base, the compound of Formula I;

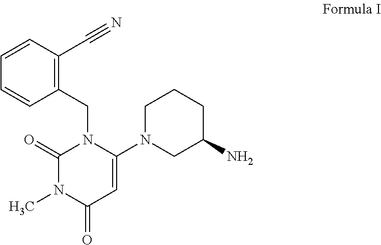 [SMILES: Cn1c(=O)cc(N2CCC[C@@H](N)C2)n(Cc2ccccc2C#N)c1=O]

According to another object of the invention, there is provided a process for the preparation of the benzoate salt of alogliptin, the compound of Formula II; comprising the steps of:
i. reacting alogliptin free base, the compound of Formula I prepared by the process as described herein, with an acid to isolate acid addition salt of alogliptin; and
ii. treating the acid addition salt of alogliptin with a base followed by reacting with benzoic acid in the presence of a solvent to isolate alogliptin benzoate salt, the compound of Formula II.

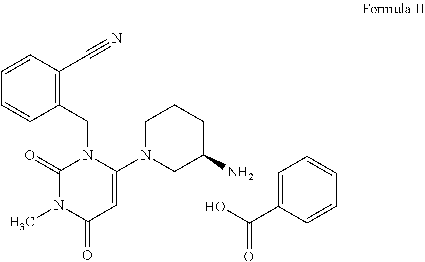

These and the other objects of the present invention will be apparent from the following detailed description.

### DETAILED DESCRIPTION OF THE INVENTION

The present invention relates to a process for preparing alogliptin, the compound of Formula I and its benzoate salt, the compound of Formula II.

In an aspect, the present invention relates to a process for the preparation of alogliptin free base, the compound of Formula I, which comprises the steps of:
a) reacting N-methylbarbituric acid, the compound of Formula III;

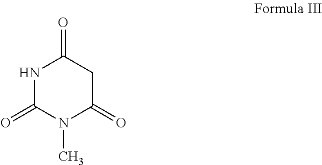

with a halogenating reagent in the presence of a base and a solvent to obtain an intermediate compound which on in situ reaction with 2-(bromomethyl)benzonitrile, the compound of Formula IV;

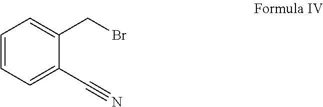

in the presence of a base and a second solvent to obtain 2-[(6-chloro-3-methyl-2,4-dioxo-3,4-dihydropyrimidin-1-(2H)-yl)methyl]benzonitrile, the compound of Formula V;

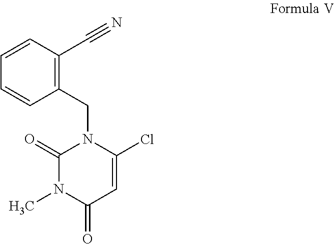 [SMILES: Cn1c(=O)cc(Cl)n(Cc2ccccc2C#N)c1=O]

b) reacting the compound of Formula V as obtained in step (a) above with (R)-piperidin-3-amine free base or its salt, the compound of Formula VI

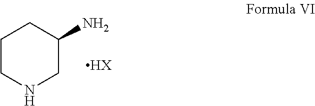

(wherein X is Cl or Br);
in the presence of an organic solvent to obtain alogliptin free base, the compound of Formula I.

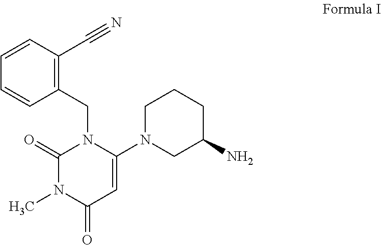 [SMILES: Cn1c(=O)cc(N2CCC[C@@H](N)C2)n(Cc2ccccc2C#N)c1=O]

The process as described above further comprises treating alogliptin free base, the compound of Formula I with an acid to isolate acid addition salt of alogliptin; which is treated with a base followed by treating it with benzoic acid to obtain the corresponding benzoate salt of alogliptin, the compound of formula II.

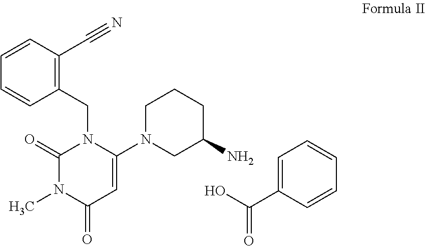

In another aspect, the present invention relates to a process for the preparation of alogliptin benzoate salt, the compound of Formula II, which comprises the steps of:
a) reacting N-methylbarbituric acid, the compound of Formula III with a halogenating reagent in the presence of a base and a solvent to obtain an intermediate compound which is reacted in situ with 2-(bromomethyl)benzonitrile, the compound of Formula IV in the presence of a base and a second solvent to obtain 2-[(6-chloro-3-methyl-2,4-dioxo-3,4-dihydropyrimidin-1-(2H)-yl)methyl]benzonitrile, the compound of Formula V;
b) reacting the compound of Formula V as obtained in step (a) above with (R)-piperidin-3-amine free base or its salt, the compound of Formula VI in the presence of an organic solvent to obtain alogliptin free base, the compound of Formula I;
c) in situ reacting alogliptin free base, the compound of Formula I as obtained in step (b) with an acid to obtain acid addition salt of alogliptin; and
d) reacting the acid addition salt as obtained in step (b) above with a base followed by reacting with benzoic acid to obtain alogliptin benzoate, the compound of Formula II.

The term “in situ” is used herein to mean that within the reaction mixture and without isolation of the intermediate compound.

In an embodiment of the present invention, the halogenating reagent used in step (a) of the process is selected from the group consisting of phosphorous oxychloride, phosphorous trichloride, phosphorous pentachloride, thionyl chloride, oxalyl chloride and phosphorous tribromide.

In an embodiment of the present invention, the halogenating reagent used in step (a) of the process is phosphorous oxychloride.

In an embodiment of the present invention, the reaction of step (a) is carried out in the presence of the solvent selected from the group consisting of toluene, xylene, dichloromethane, dichloroethane, and chlorobenzene or a mixture thereof.

In an embodiment of the present invention, the reaction of step (a) is carried out at a suitable temperature, preferably at a temperature ranging from 30° C. to the reflux temperature of the solvent used for the reaction. The most preferred temperature of the reaction is in the range of 85° C. to 105° C.

In an embodiment of the present invention, the base used in the reaction step (a) is an organic base selected from the group consisting of N,N-dimethylaniline, pyridine, morpholine, tertiary-butylamine and triethylamine.

In an embodiment of the present invention, after completion of the reaction of step (a), the reaction mixture is cooled to about 10° C., and the resulting reaction mass is diluted with a first solvent selected from the group consisting of methanol, ethanol, isopropyl alcohol, n-butanol, tertiary-butanol and stirred for 1-2 hour. The liquid from the reaction mass is separated and to the residual mass of the intermediate compound, second solvent selected from the group consisting of tetrahydrofuran, N,N-dimethylformamide, dimethylsulfoxide, N-methylpyrrolidine and toluene, is added. The solution of the intermediate compound is further reacted with 2-(bromomethyl)benzonitrile, the compound of Formula IV in the presence of a base at a temperature range of 55° C. to 90° C. for 4 to 5 hours. The base used for this reaction is selected from the group consisting of N,N-dimethylaniline, pyridine, morpholine, tertiary-butylamine, diisopropylethylamine and triethylamine. The completion of the reaction is monitored by HPLC technique. After completion of the reaction, the solvent is concentrated under reduced pressure to get a residual solid. To the residual solid charged mixture of solvent selected from water methanol, ethanol, isopropanol, n-butanol and tertiary-butanol and is stirred at 10° C. to 20° C. for one hour. The preferred mixture of solvent selected is water and isopropanol. The solid that is separated out is filtered and washed with isopropanol to obtain pure 2-[(6-chloro-3-methyl-2,4-dioxo-3,4-dihydropyrimidin-1-(2H)-yl)methyl]benzonitrile, the compound of Formula V.

In an embodiment of the present invention, in the reaction of step (b), the compound of Formula V, 2-[(6-chloro-3-methyl-2,4-dioxo-3,4-dihydropyrimidin-1-(2H)-yl)methyl]benzonitrile as obtained in the above step (a) is condensed with (R)-piperidin-3-amine free base or its salt, the compound of Formula VI in the presence of a base, a solvent and optionally in the presence of a suitable catalyst to obtain the desired alogliptin free base, the compound of Formula I.

In an embodiment of the present invention, the base used in the condensation reaction of step (b) is selected from the group consisting of sodium carbonate, potassium carbonate, sodium bicarbonate, potassium bicarbonate, triethylamine and diisopropylethylamine.

In an embodiment of the present invention, the solvent used in the condensation reaction of step (b) is a protic or an aprotic solvent selected from the group consisting of methanol, isopropanol, ethyl acetate, N,N-dimethylformamide, dimethylsulfoxide and N,N-dimethylacetamide; or a mixture thereof. In a preferred embodiment, a mixture of two to three solvents is used in the step (b).

In an embodiment of the present invention, the reaction of step (b) is carried out in the presence of a catalyst selected from potassium iodide, sodium iodide, ammonium iodide and tetrabutylammonium iodide. In a preferred embodiment, the catalyst used for the reaction is potassium iodide.

In an embodiment of the present invention, the reaction of step (b) is carried out at a temperature in the range of 50° C. to 90° C. In a preferred embodiment, the reaction is carried out at 65° C. to 75° C.

In another embodiment of the present invention, the present invention relates to a process for the preparation of alogliptin benzoate salt, the compound of Formula II, comprising the steps of
i. reacting alogliptin free base the compound of Formula I, obtained according to the process of the present invention with an acid to isolate acid addition salt of alogliptin;
ii. treating the acid addition salt of alogliptin with a base followed by reacting with benzoic acid in the presence of a suitable solvent to isolate alogliptin benzoate salt, the compound of Formula II.

In an embodiment of the present invention, the acid addition salt is selected from the group consisting of hydrochloric acid, sulphuric acid, phosphoric acid and acetic acid. The preferred acid addition salt is hydrochloric acid salt.

In an embodiment of the present invention, the base used in the reaction is selected from the group consisting of sodium carbonate, potassium carbonate, sodium bicarbonate, potassium bicarbonate, triethylamine and diisopropylethylamine. The preferred base used for the reaction is sodium carbonate.

In an embodiment of the present invention, the solvent used in the reaction involving treatment with benzoic acid is selected from the group consisting of methanol, ethanol, 1-propanol, isopropyl alcohol, n-butanol, 2-butanol, tertiary-butanol, ethyl acetate, acetone, methyl ethyl ketone, methyl isobutyl ketone, dichloromethane and cyclohexane; or a mixture thereof.

The isolated alogliptin benzoate salt can be further purified by any conventional purification method known to a person skilled in the art to obtain pure alogliptin benzoate, the compound of Formula II.

The starting material, N-methylbarbituric acid, the compound of Formula III used in the process of the present invention is a readily available commercial compound, and is also relatively cheap in terms of its cost. Thus, use of N-methylbarbituric acid as the starting material renders the process of the present invention cost-effective.

Also, the process of the present invention is advantageous because it avoids use of metal hydride such as sodium hydride as a base thereby rendering the process industrially safe.

The following examples, which fully illustrate the practice of the preferred embodiments of the present invention, are intended to be for illustrative purpose only, and should not be considered to be limiting to the scope of the present invention.

### EXAMPLES

### Example 1: Preparation of 2-[(6-chloro-3-methyl-2,4-dioxo-3,4-dihydropyrimidin-1-(2H)-yl)methyl]benzonitrile (the Compound of Formula V)

In a dry flask charged toluene (150 ml), N-methylbarbituric acid (50 gm), phosphorous oxychloride (64.5 gm) and N,N-dimethylaniline (20 ml). Heated the reaction mass to 90° C.-95° C. and maintained the reaction at this temperature for 2 hours. The reaction progress was monitored by TLC. After completion of the reaction, cooled the reaction mass to 10° C.-15° C. and charged methanol (150 ml) in the reaction mass and stirred at this temperature for 1 hour. The reaction mass was allowed to settle and the solvent was removed from the reaction mass. Charged again methanol (50 ml), stirred and separated the solvent from the reaction mixture to get the residual solid mass of the intermediate compound.

To the solution of the intermediate compound charged solvent tetrahydrofuran (300 ml), 2-(bromomethyl)benzonitrile (67.3 gm) and diisopropylethylamine (60.5 gm). Raised the temperature of the reaction mass to 60° C.-65° C. and maintained at this temperature for 4-5 hours. The reaction progress was monitored by TLC. After the completion of the reaction the solvent was concentrated and to the residual mass charged water (300 ml) and stirred the reaction mass at 25° C.-30° C. for 1 hour. Filtered the reaction mass and washed the solid mass with water (50 ml). The filtered wet solid mass was taken in solvent isopropyl alcohol (150 ml) and stirred at 25° C.-30° C. for 1 hour. Filtered the solid and washed with isopropyl alcohol (50 ml) and dried the compound to get 2-[(6-chloro-3-methyl-2,4-dioxo-3,4-dihydropyrimidin-1-(2H)-yl)methyl]benzonitrile. Dry weight=52 gm.

### Example 2: Preparation of 2-[(6-chloro-3-methyl-2,4-dioxo-3,4-dihydropyrimidin-1-(2H)-yl)methyl]benzonitrile (the Compound of Formula V)

In a dry flask charged toluene (250 ml), N-methylbarbituric acid (50 gm), phosphorous oxychloride (86.4 gm) and N,N-dimethylaniline (21.3 gm). Heated the reaction mass to 85° C.-90° C. and maintained the reaction at this temperature for 5 hours. The reaction progress was monitored by HPLC. After completion of the reaction, cooled the reaction mass to 0° C.-5° C. and charged methanol (200 ml) in the reaction mass and stirred at this temperature for 1 hour. The reaction mass was allowed to settle and siphon out solvent from the reaction mass. Charged again methanol (50 ml) stirred and separated the solvent from the reaction mixture to get the residual mass of the intermediate compound.

To the solution of the intermediate compound, charged solvent tetrahydrofuran (200 ml), 2-(bromomethyl)benzonitrile (55.18 gm) and diisopropylethylamine (74.9 ml). Raised the temperature of the reaction mass to 65° C.-70° C. and maintained at this temperature for 4-5 hours. The reaction progress was monitored by HPLC. After the completion of the reaction the solvent was concentrated and to the residual mass charged mixture of solvents, water and IPA and stirred the reaction mass at 10° C.-15° C. for 1 hour. Filtered the reaction mass and washed the solid mass with IPA (50 ml), dried the compound to get 2-[(6-chloro-3-methyl-2,4-dioxo-3,4-dihydropyrimidin-1-(2H)-yl)methyl]benzonitrile, the compound of Formula V.

Dry weight=40 gm.

### Example 3: Preparation of 2-[[6-[(3R)-3-amino-1-piperidinyl]-3,4-dihydro-3-methyl-2,4-dioxo-1(2H)-pyrimidinyl]methyl]benzonitrile Hydrochloride (Alogliptin Hydrochloride)

In a dry flask charged isopropyl alcohol (250 ml), 2-[(6-chloro-3-methyl-2,4-dioxo-3,4-dihydropyrimidin-1-(2H)-yl)methyl]benzonitrile (50 gm), (R)-3-aminopiperidine dihydrochloride (35.16 gm), sodium carbonate (48.06 gm) and potassium iodide (2.50 gm). Heated the reaction mass to 65° C.-70° C. and maintained the reaction at this temperature for 12 to 14 hours. The reaction progress was monitored by HPLC. After completion of the reaction, cooled the reaction mass to 45° C.-50° C. then filtered it at same temperature followed by isopropyl alcohol (50 ml) wash. Collected the filtrate and concentrated undervacuum at 45° C.-50° C. to obtain the residual mass. To this residual mass charged dichloromethane (350 ml) to get the clear solution.

Washed dichloromethane layer using purified water (250 ml×3) and concentrated solvent maintaining temperature between 38° C. to 40° C. to remove one volume of solvent quantity charged. Applied cooling and cooled the reaction mass to 25° C. to 30° C. Charged slowly the acidic solution of isopropanol hydrochloride maintaining the temperature of the reaction mass between 25° C. to 30° C. to bring the pH to less than 2. Stirred the reaction mass at 25° C.-30° C. for 2 hours. Filtered the reaction mass and washed the wet cake with dichloromethane and dried the product to get 2-[[6-[(3R)-3-amino-1-piperidinyl]-3,4-dihydro-3-methyl-2,4-dioxo-1(2H)-pyrimidinyl]methyl]benzonitrile hydrochloride (alogliptin hydrochloride).

Dry weight=40 gm.

### Example 4: Preparation of Alogliptin Benzoate Salt (the Compound of Formula II)

In a flask, charged purified water (250 ml), alogliptin hydrochloride (50 gm), sodium carbonate solution (15.5 gm of sodium carbonate in 77.50 ml purified water) and stirred for 30 minutes to get clear solution. Maintain the pH of the reaction solution between 8 to 10, and extract the solution with dichloromethane. Separated the dichloromethane layer and washed the layer with purified water. After charcoalisation and filtering through hyflow the solvent was concentrated at 35° C. to 40° C.

Charged solvent 1-propanol (175 ml) and solution of benzoic acid (16.25 μm) in 1-propanol (100 ml) to the reaction mass at 40° C. to 45° C. to get clear solution which is stirred and maintained the reaction at 55° C. to 60° C. for 2.0 hours. Cooled the reaction mass to 25° C. to 30° C. and maintained for one hour. Filtered the reaction mass and washed the solid mass with 1-propanol, dried the compound to obtain pure alogliptin benzoate the compound of Formula II.

Dry weight=42.50 gm.

1. A process for the preparation of alogliptin, the compound of Formula I, and its pharmaceutically acceptable salt thereof, 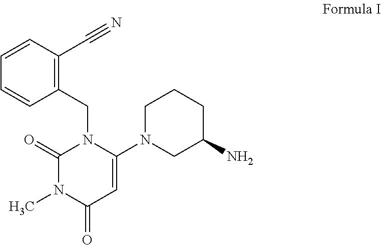 [SMILES: Cn1c(=O)cc(N2CCC[C@@H](N)C2)n(Cc2ccccc2C#N)c1=O]
which comprises the steps of:
a) reacting N-methylbarbituric acid, the compound of Formula III; 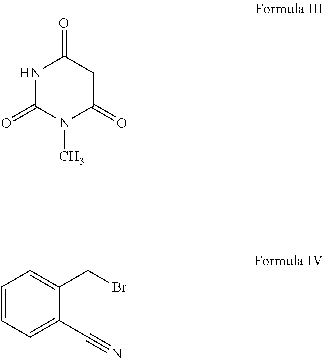
with a halogenating reagent in the presence of a base and a solvent to get an intermediate compound which undergoes an in situ condensation reaction with 2-(bromomethyl)benzonitrile, the compound of Formula IV in the presence of a base and a second solvent to obtain 2-[(6-chloro-3-methyl-2,4-dioxo-3,4-dihydropyrimidin-1-(2H)-yl)methyl]benzonitrile, the compound of Formula V; 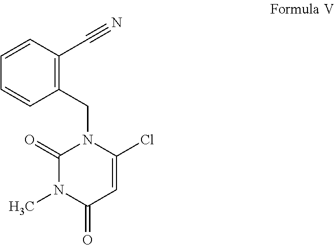 [SMILES: Cn1c(=O)cc(Cl)n(Cc2ccccc2C#N)c1=O]
b) reacting the compound of Formula V obtained in step (a) with (R)-piperidin-3-amine free base or its salt, the compound of Formula VI; 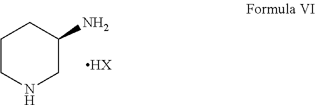
wherein X is Cl or Br;
in the presence of an organic solvent and a base to obtain alogliptin, the compound of Formula I and
c) reacting the compound of formula I obtained in step (b) with an acid in the presence of a solvent to obtain the corresponding acid addition salt of alogliptin.

2. The process according to claim 1, wherein the halogenating reagent used in step (a) of the process is selected from the group consisting of phosphorous oxychloride, phosphorous trichloride, phosphorous pentachloride, thionyl chloride, oxalyl chloride and phosphorous tribromide.

3. The process according to claim 2, wherein the halogenating reagent is phosphorous oxychloride.

4. The process according to claim 1, wherein the solvent used in the reaction of N-methylbarbituric acid, the compound of Formula III with halogenating agent in step (a) is selected from the group consisting of toluene, xylene, dichloromethane, dichloroethane, chlorobenzene, and a mixture thereof.

5. The process according to claim 1, wherein the reaction of step (a) is carried out at a temperature ranging from 30° C. to the reflux temperature of the solvent used.

6. The process according to claim 5, wherein the reaction of step (a) is carried out at temperature ranging from 85° C. to 105° C.

7. The process according to claim 1, wherein the base used in the reaction step (a) is an organic base selected from the group consisting of N,N-dimethylaniline, pyridine, morpholine, tertiary-butylamine, diisopropylethylamine and triethylamine.

8. The process according to claim 1, wherein the second solvent used in the condensation reaction of step (a) is selected from the group consisting of tetrahydrofuran, N,N-dimethylformamide, dimethylsulfoxide, N-methylpyrrolidine and toluene.

9. The process according to claim 1, wherein the base used in the condensation reaction of step (a) is selected from the group consisting of sodium carbonate, potassium carbonate, sodium bicarbonate, potassium bicarbonate, triethylamine and diisopropylethylamine.

10. The process according to claim 1, wherein the organic solvent used in the reaction of step (b) is a protic or an aprotic solvent selected from the group consisting of methanol, isopropanol, ethyl acetate, N,N-dimethylformamide, dimethylsulfoxide, N,N-dimethylacetamide, and a mixture thereof.

11. The process according to claim 1, wherein the reaction of step (b) is carried out in the presence of a catalyst selected from the group consisting of potassium iodide, sodium iodide, ammonium iodide and tetrabutylammonium iodide.

12. The process according to claim 11, wherein the catalyst used is potassium iodide.

13. The process according to claim 11, wherein the reaction of step (b) is carried out at a temperature in the range of 50° C. to 90° C.

14. The process according to claim 1, wherein in step (c), the acid is benzoic acid and the acid addition salt is alogliptin benzoate.

15. A process for the preparation of alogliptin benzoate salt, the compound of Formula II, comprising the steps of:
a) preparing an acid addition salt of alogliptin by the process of claim 1; and
b) treating the acid addition salt of alogliptin with a base to obtain a free base, followed by reacting the free base with benzoic acid in the presence of a solvent to isolate alogliptin benzoate salt, the compound of Formula II.

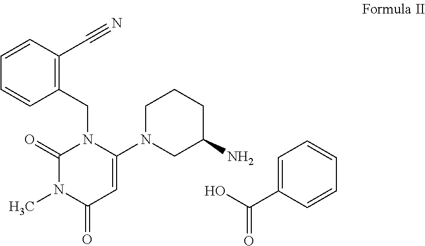

16. The process according to claim 15, wherein the acid addition salt is selected from the group consisting of a hydrochloric acid addition salt, a sulphuric acid addition salt, a phosphoric acid addition salt, and an acetic acid addition salt.

17. The process according to claim 15, wherein the base used in the step of treating the acid addition salt of alogliptin is selected from the group consisting of sodium carbonate, potassium carbonate, sodium bicarbonate, potassium bicarbonate, triethylamine and diisopropylethylamine.

18. The process according to claim 15, wherein the solvent used for reacting the free base with benzoic acid is selected from the group consisting of methanol, ethanol, 1-propanol, isopropyl alcohol, n-butanol, 2-butanol, tertiary-butanol, ethyl acetate, acetone, methyl ethyl ketone, methyl isobutyl ketone, dichloromethane and cyclohexane, and a mixture thereof.
